(19) United States
(12) Patent Application Publication
(10) Pub. No.: US 2015/0196376 A1
(43) Pub. Date: Jul. 16, 2015

(54) Implants with Anti-Migration Features and/or Members for Stress Urinary Incontinence Treatments and Related Methods

(71) Applicant: Urova Medical, Inc., Memphis, TN (US)

(72) Inventors: Franklin Yao, New York, NY (US); Adam Xiao, Fallston, MD (US); Kelsey Humphries, Boise, ID (US); Shida Li, West Chester, PA (US); Michael Chen, Portland, OR (US); Amanda Ojeda, Easton, PA (US); Howard Logan, Surfside Beach, SC (US); Vidhan Agrawal, Memphis, TN (US)

(21) Appl. No.: 14/409,706

(22) PCT Filed: Jun. 25, 2013
(86) PCT No.: PCT/US2013/047586

§ 371 Date: Dec. 19, 2014

Related U.S. Application Data
(60) Provisional application No. 61/665,047, filed on Jun. 27, 2012.

(51) Int. Cl. A61F 2/00; B29C 45/00; A61B 17/00

(52) U.S. Cl. CPC A61F 2/0045; A61B 17/00234; B29C 45/00; A61F 2240/001; A61F 2250/0078; B29L 2031/7532

(57) ABSTRACT

An urogynecologic implant has a curved body that disperses force and reduces the ability of the urethra to expand into the pelvic floor under impulses of abdominal pressure in order to inhibit, reduce or prevent stress urinary incontinence.

### RELATED APPLICATIONS

This application claims the benefit of and priority to U.S. Provisional Application Ser. No. 61/665,047, filed Jun. 27, 2012, the contents of which are hereby incorporated by reference as if recited in full herein.

### FIELD OF THE INVENTION

The present invention relates to surgical implants to treat urinary incontinence.

### BACKGROUND

Stress incontinence is the involuntary leakage of urine due to increased abdominal pressure such as during a cough. This is particularly prevalent in women and has been shown to degrade the quality of life. In the past, transvaginal slings, transobturator tape, and a single-incision mini-slings have been used to attempt to treat this condition. Generally stated, these devices include a surgical mesh that is placed around the mid-urethra to form a hammock-like structure, anchoring it to the pubic bone to attempt to prevent the leakage of urine.

However, the transvaginal and transobturator slings require three incisions and the surgery is performed in a hospital operating room, making it inconvenient and expensive. The mini-sling only requires a single incision, but it has been associated with a lower effectiveness and a higher risk of complications, which have recently come under FDA review.

### SUMMARY OF EMBODIMENTS OF THE INVENTION

Embodiments of the present invention provide minimally invasive surgical implants that can inhibit or prevent stress urinary incontinence.

The implants can be placed and/or implanted to operate in a non-restrictive way to increase the ease of implantation and prevent voiding complications.

Embodiments of the invention are directed to three dimensional rigid or semi-rigid urogynecologic implants having a defined three dimensional self-supporting shape sized and configured to reside between a urethra and an anterior vaginal wall of a patient. The implants can have a plurality of anti-migration features and/or members.

Embodiments of the invention are directed to stress incontinence implants that include a three dimensional rigid or semi-rigid urogynecologic implant body having a defined three dimensional self-supporting shape sized and configured to reside between a urethra and an anterior vaginal wall of a patient. The implant body includes a plurality of anti-migration members.

The anti-migration members can include outwardly extending arms.

The anti-migration members can include surface protrusions.

The implant body can have at least one embossed primary surface.

The anti-migration members can include flexible outwardly extending arms that are configured to have sufficient flexibility to flex toward or against the implant body when compressed during placement in the body and sufficient rigidity to translate to a fully extended self-supporting configuration in position in a respective patient.

The implant body can have four corners. The anti-migration members can include at least one flexible outwardly extending arm that resides on each corner. The arms can be configured to have sufficient flexibility to flex toward or against the implant body when compressed during placement in the body and sufficient rigidity to translate to a fully extended self-supporting configuration in position in a respective patient.

The arms can have a first segment that merges into a second segment that has a free outer end. The second segment can extend at an angle that is between 15-90 degrees with respect to a centerline of the first segment.

The implant body can have four (or more) corners.

The anti-migration members include surface protrusions at least some of which can reside proximate the corners.

The implant body can include a first primary surface that is adapted to contact an outerwall of the urethra and a second primary surface underlying the first primary surface adapted to contact an anterior vaginal wall. The anti-migration members can include arms that reside closer to the second primary surface adjacent the anterior vaginal wall.

The implant body and anti-migration members can be a molded monolithic body or an integral body with one of the implant body or the anti-migration members having a greater hardness than the other.

The implant body can have a Young's modulus between about 2 MPa and about 10 MPa

The implant body can have a width dimension and a length dimension, with the width dimension being about the same or less than the length dimension.

The implant body can include a first primary surface that is adapted to contact an outerwall of the urethra and a second primary surface underlying the first primary surface adapted to contact an anterior vaginal wall. The first primary surface can have medial region that is raised and has an increased thickness relative to outer end portions thereof. The anti-migration members can (i) reside on or extend from the outer end portions thereof or can (ii) includes both arms that extend outwardly from the outer end portions and surface protrusions that extend upwardly from the outer end portions.

The implant body can be sized and configured to surround only about 180 degrees or less of a female urethra and can have a maximum thickness that is between about 1 mm to about 5 mm.

Other embodiments are directed to surgical tools for placing a urinary stress incontinence implant in a female patient. The tools include a surgical tool with a cavity; and a three-dimensional rigid or semi-rigid shaped implant body sized and configured to reside between a urethra and anterior vaginal wall of a female patient, the implant having a plurality of anti-migration arms. The implant body is held in the cavity with the anti-migration arms held proximate the implant body. The tool is configured to slidably release the implant body from the cavity so that the arms automatically expand outwardly as they exit the tool cavity to engage local tissue.

The cavity can include an aperture that slidably receives a pusher and the pusher can be extendable to push the implant body out of the cavity to automatically deploy the arms.

Yet other embodiments are directed to methods of fabricating a urinary incontinence implant. The methods include forming a rigid or semi-rigid three dimensional implant body with anti-migration members, the implant body having sufficient rigidity to define a self-supporting three-dimensional shape with attached anti-migration members.

The forming step can be carried out by molding the implant body and the anti-migration members. The anti-migration members can include at least one of surface protrusions proximate four corners of a first primary surface or four outwardly extending legs extending off four corners of a second underlying primary surface.

Still other embodiments are directed to methods of implanting a stress urinary incontinence implant. The methods include: (a) providing a surgical tool with a cavity having an open forward end releasably holding an implant body with flexible anti-migration arms extending outwardly from the implant held against sidewalls of the cavity to force the arms toward the implant body; and (b) releasing the implant body from the cavity of the tool at a pocket location between a urethra and vaginal wall and allowing the arms to return to an outwardly extended configuration to engage local tissue thereat.

The arms can reside on respective corner portions of the implant body. The implant body can optionally include surface protrusions extending off a first primary surface to face the urethra.

The method can include forming pores in the implant body during or after the molding.

The forming the pores can include directing laser light into the molded implant body to form through channels.

Before the forming step, the method can include providing a flowable material of the moldable material that can be combined with a porogen in the mold or prior to introducing into the mold, then after the forming step, the porogon can be removed from the molded implant body leaving a porous implant body.

The forming step can include injection molding implant material in a mold having a cavity that defines the first and second radii of curvature.

The implant material can include silicone.

Still other embodiments are directed to molds for a medical stress incontinence implant. The molds include a mold body having an internal volumetric cavity with walls that are configured with first and second radii of curvature that are configured to form a rigid or semi-rigid three dimensional implant body having a three-dimensional shape including a first radius of curvature associated with a medial portion of a first primary surface and a second radius of curvature associated with an arc of an underlying second primary surface.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

Other systems and/or methods according to embodiments of the invention will be or become apparent to one with skill in the art upon review of the following drawings and detailed description. It is intended that all such additional systems, methods, and/or devices be included within this description, be within the scope of the present invention, and be protected by the accompanying claims.

### BRIEF DESCRIPTION OF THE DRAWINGS

Other features of the present invention will be more readily understood from the following detailed description of exemplary embodiments thereof when read in conjunction with the accompanying drawings.

FIG. 1 is a top perspective view of one example of a surgical implant according to embodiments of the present invention.

FIG. 2 is a side view along one long edge of the implant shown in FIG. 1.

FIG. 3 is a side view along one short edge of the implant shown in FIG. 1.

FIG. 4 is a top view of the implant shown in FIG. 1.

FIG. 5 is a top perspective view illustrating an exemplary implant with mesh attachments according to some embodiments of the present invention.

FIGS. 6A-6C are schematic illustrations of a response of the pubourethral system to a cough without the present invention.

FIGS. 7A-7C are schematic illustrations of the response of the pubourethral system to a cough with the present invention according to embodiments of the present invention.

FIG. 8 is an enlarged schematic illustration of an implant in position according to embodiments of the present invention.

FIGS. 9A-9C are schematic illustrations of a sequence of surgical steps that can be used to place urogynecologic implants according to embodiments of the present invention.

FIGS. 10A-10C are illustrations of exemplary dimensions and radii according to particular embodiments of the present invention.

FIG. 11 is a side section view (taken inward of one long edge) of the implant shown in FIG. 1 illustrating a hollow interior cavity according to embodiments of the present invention.

FIG. 12 is a side view (along one long edge) of an exemplary implant illustrating different end configurations and that the implant may include one or more pores or apertures according to embodiments of the present invention.

FIG. 13 is a perspective view of an implant similar to that shown in FIG. 1 illustrating that the implant can have sufficient pores to allow fluid transport therethrough according to embodiments of the present invention.

FIG. 14 is a schematic illustration of different size implants selected to accommodate different size urethras according to embodiments of the present invention.

FIG. 15 is a schematic illustration of different size implants selected to accommodate different degrees of severity of urinary stress incontinence according to embodiments of the present invention.

FIG. 16A is a side perspective view of an exemplary mold for fabricating an implant according to embodiments of the present invention.

FIG. 16B is a section view of the mold shown in FIG. 16A according to embodiments of the present invention.

FIG. 17 is a schematic illustration of a method/system for forming pores in a stress incontinence implant according to some embodiments of the present invention.

FIG. 18 is a schematic illustration of another embodiment of a method/system for forming pores in a stress incontinence implant according to some embodiments of the present invention.

FIG. 19A is a top view of an implant according to embodiments of the present invention.

FIG. 19B is an end view of the implant shown in FIG. 19A according to embodiments of the present invention.

FIG. 19C is a top view of another embodiment of an implant according to embodiments of the present invention.

FIG. 20A is a top view of yet another embodiment of an implant according to embodiments of the present invention.

FIG. 20B is a side view of the implant shown in FIG. 20A.

FIG. 20C is a top view of yet another embodiment of an implant similar to that shown in FIG. 20A but with a greater length, according to embodiments of the present invention.

FIG. 20D is an enlarged schematic illustration of different exemplary surface projection features according to embodiments of the present invention.

FIGS. 21A-21F are schematic illustrations of different exemplary configurations of free end portions of arms according to embodiments of the present invention.

FIGS. 22A-22D are schematic illustrations of an exemplary sequence of actions that can be carried out using a surgical tool to place an implant according to embodiments of the present invention.

### DETAILED DESCRIPTION OF EMBODIMENTS OF THE INVENTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise. One or more features shown and discussed with respect to one embodiment may be included in another embodiment even if not explicitly described or shown with another embodiment.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

The term “about” means that the recited number or value can vary by +/−20%.

The term “sterile” means that the noted device or material meets or exceeds defined medical guidelines of cleanliness and is substantially (if not totally) without contaminants so as to be suitable for medical uses.

The term “urogynecologic implant” refers to implants targeted for use between a urethra and vagina of female patients. The implants may be acutely or chronically placed. The implants may be for medical or veterinarian uses, e.g., for human or animals, but are particularly suitable for human use.

In contrast to conformally configured slings, the term “semi-rigid” means that the implant is flexible, typically elastically flexible in at least one dimension, but has sufficient rigidity to be self-supporting and able to maintain its three-dimensional shape outside a target body in a non-loaded, free-standing configuration. The implant can also be configured so as to be able to substantially maintain a non-loaded pre-implanted 3-D external shape while in position in the body, typically even when under normal, non-stress related loading in the body.

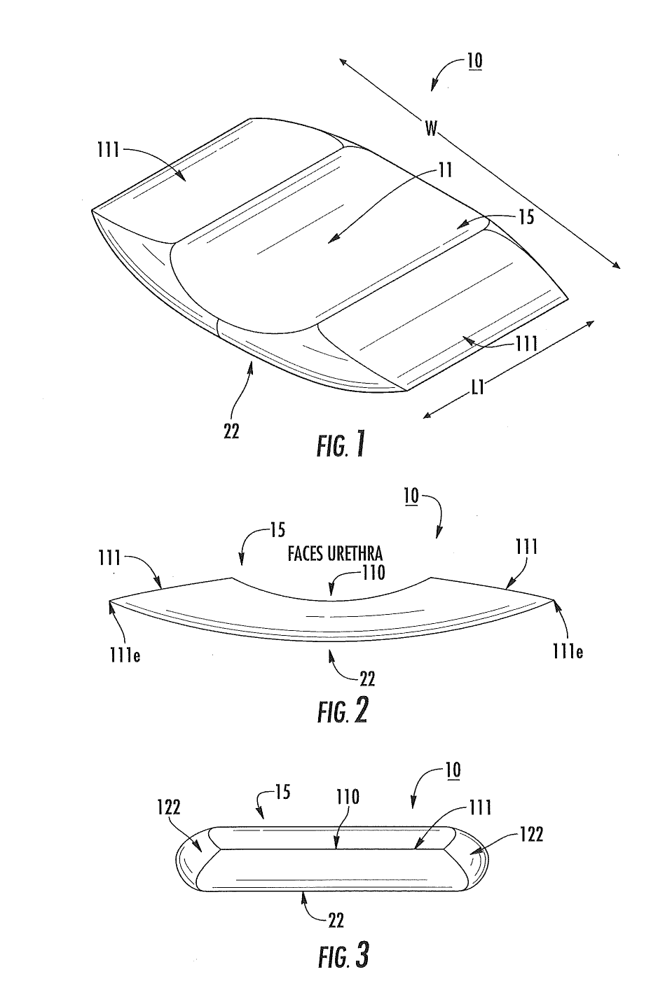

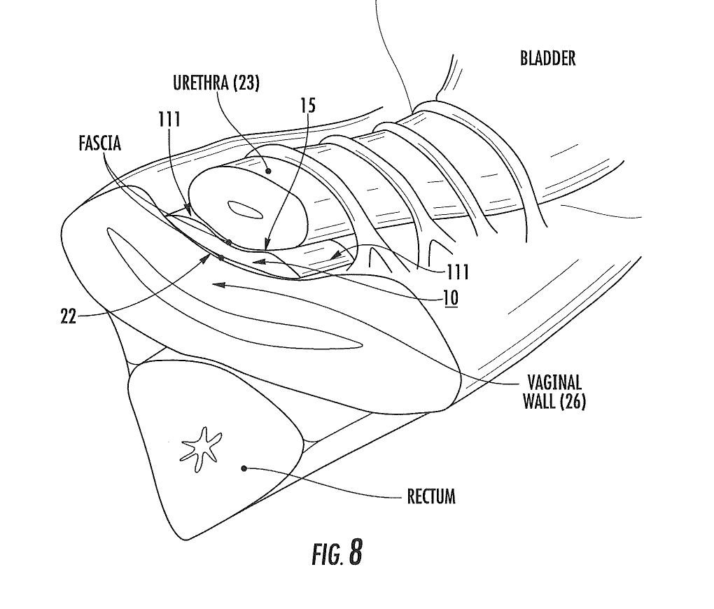

Referring now to FIGS. 1-4, there is shown an implant 10 having a rigid or semi-rigid curved shape. The implant 10 has two opposing primary surfaces 15, 22. The first primary surface 15 can be curved, typically in a direction facing a urethra 23 (FIGS. 7A, 8). In the orientation shown, the first primary surface 15 is a top surface. The first primary surface 15 can include a medial portion that is curved or has a relatively wide groove 110 that merges into two downwardly-curved or linearly tapered end portions 111 that can terminate at an outermost edge 111e (which may be sharp, blunt or rounded). The other primary surface 22 can also be upwardly curved (or curved in a direction facing the urethra 23) and meet the upper primary surface 15 at the edge 111e. The second primary surface 22 may also be substantially planar (not shown). Other configurations of the implant may be used.

The implant 10 can have a width “W” that is about the size of a diameter of an average urethra (e.g., about 20 mm). In some embodiments, the length dimension “L” can be less than the W dimension, such as between about 20-60% less, such as about 50% less. In some embodiments, the length dimension L can be greater than width dimension. In particular embodiments, the length L can be between about 7 to about 30 mm

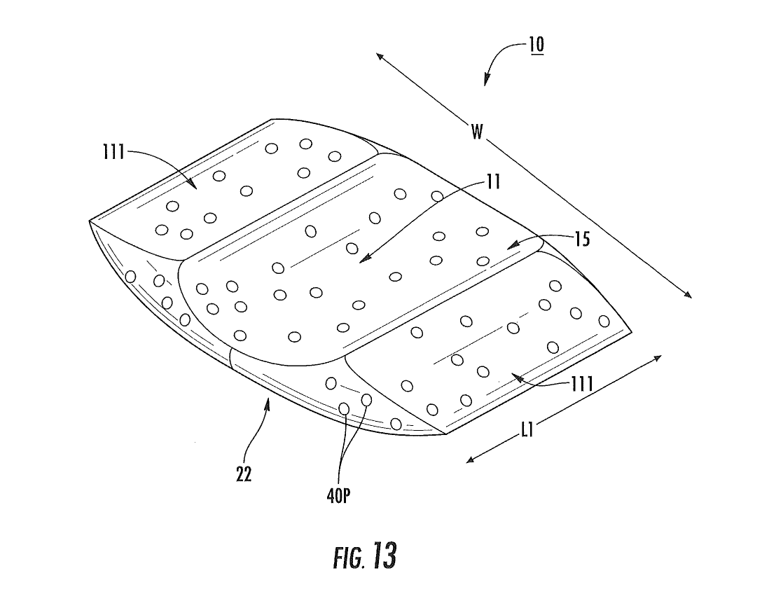

The length L1 (at the ends) can be about 10 mm to about 20 mm. The maximum length dimension L2 may be at the center and be greater than the length L1 at the ends 111 as shown in FIG. 10C and this may be about 14 mm to about 24 mm In some particular embodiments, the length L1 can be about ⅓ the length of an average lower end length of a urethra.

The maximum thickness (average) can be between about 1 mm to about 10 mm, typically 1 mm to 9 mm, including between about 1-6 mm, such as about 1. 5 mm, about 2 mm, about 2.5 mm, about 3 mm, about 3.5 mm, about 4 mm, about 4.5 mm or about 5 mm.

The surface area of the curved surface or groove 110 can be about 50% of the longer surface 22 as shown in FIGS. 1 and 10C. However, this relationship can vary and it is contemplated that a less incontinent woman may use an implant where the groove or curved surface 110 is closer in area to the longer second primary surface 22 while a more incontinent woman may use an implant where the shorter curved or groove surface 110 has an area that is even less than about 50% than the longer surface to provide increased support.

In some embodiments, the implant 10 can be sized and configured to surround only about 180 degrees or less of a female urethra, typically between about 60-120 degrees, and can have a maximum thickness that is between about 1 mm to about 6 mm, typically about 1.5 mm to about 5 mm, and in some embodiments about 2 mm, about 2.5 mm, about 3 mm, about 3.5 mm, about 4 mm, about 4.5 mm or about 5 mm.

In some embodiments, the implant 10 can have a width W that is between about 15-30 mm, a length L that is between about 7-30 mm and a maximum thickness that is between about 1 mm and about 5 mm.

FIGS. 10A-10C illustrate exemplary radii and dimensions (and reflect average dimensions) of the implant 10. As shown, the implant 10 can include three different radii, a first radius R1 defining the curvature of the groove or middle portion 110 of the upper surface 15, a second radius R2 defining the curvature of the lower or second primary surface 22 and an optional third radius R3 for defining the interface between the surfaces 15, 22. The first radius R1 can have a dimension that is about 10 mm measured from a centerline (marked as “C/L” as shown in FIG. 10A). The second radius R2 can be at least about double the first, such as about 27 mm The third radius R3, where used, can be the largest radius, typically about 30-50% greater than R2, and may be about 39.4 mm. The edge 111e may reside closer to the first primary surface 15 than the second 22, e.g., at about 1.8 mm on a side having an overall thickness of about 2 mm.

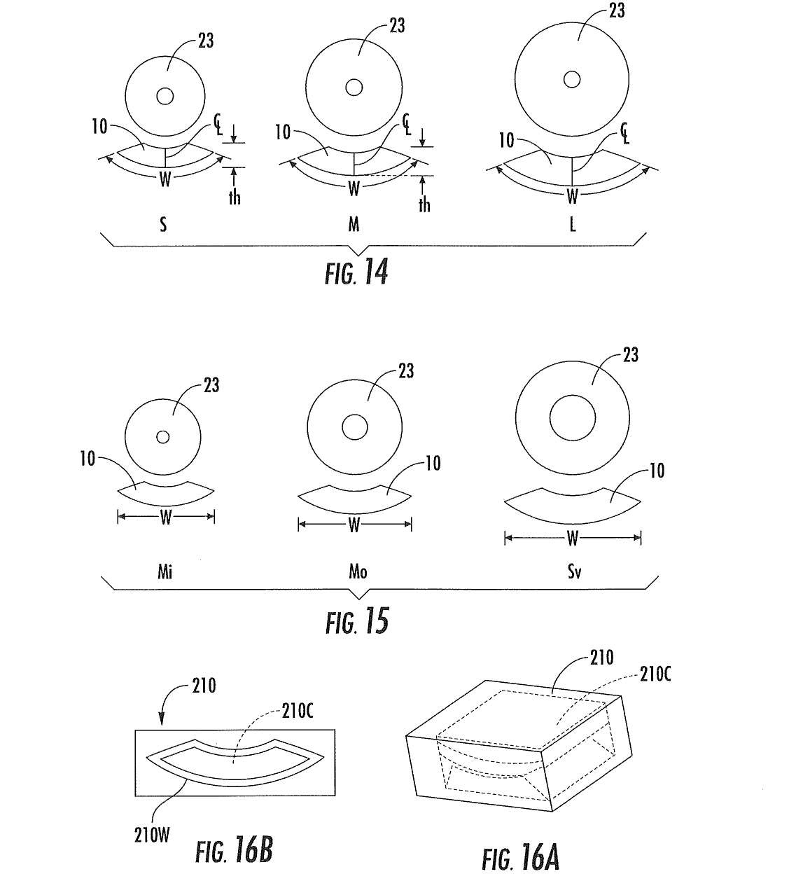

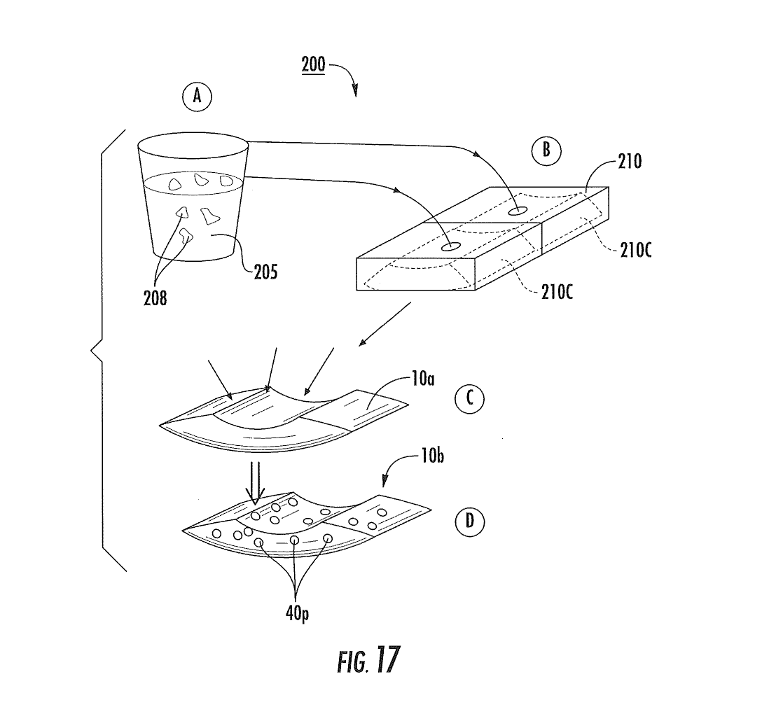

The shapes and size of the implant 10 can vary from that shown. Also, as shown in FIGS. 14 and 15, the implant 10 can be provided in a range of different sizes for different anatomical and/or functional requirements or fit. For example, the implants 10 can be provided in a plurality of defined ranges, such as “small,” “medium” and “large” with different lengths, widths and/or potentially thicknesses and/or differing degrees of rigidity. The middle portion 110 of the surface 15 may change as the ratio R of a shorter to longer surface 15:22 changes. Also, the thickness of the implant and overall shape (and rigidity) may vary according to other factors such as the degree of pelvic prolapse.

FIG. 14 illustrates that the maximum width W (the width of the longest side dimension) is typically 10-25% greater than the outer diameter of the lower to mid portion of the urethra, at the location the implant 10 is to be positioned. As shown, for a small urethra, e.g., about a 1.4 cm diameter urethra, the longest length of a suitable implant can be about 1.6 cm or about 1.7 cm; for a medium size urethra, e.g., about a 1.7 cm diameter urethra, the maximum outer length of the implant can be about 2.0 cm; and for a large urethra, e.g., about a 2.0 cm diameter urethra, a 2.4 cm implant may be appropriate. The thickness dimension (measured at the centerline) of the implant 10 can vary, including between about 2.5 mm to about 6 mm, typically between about 3 mm to about 5 mm, on average, measured at the centerline C/L.

FIG. 15 illustrates that the size/configuration of an implant 10 can be selected according to the severity of the stress urinary incontinence, e.g., mild (Mi), moderate (Mo) or severe Sv, with the width dimension W being wider in that order, such as from about 1.6 cm, to about 2.0 cm, to about 2.4 cm, or other size demarcations of increasing widths. Again, the thickness and/or rigidity of the implant may also vary according to a rated severity of the condition.

The curvatures of primary surfaces 15 and 22 can be shaped to fit about adjacent surrounding anatomy. In position, the first primary surface 15 can contact an outer surface of a lower to mid-portion of the urethra 23 (FIGS. 7A, 8) while the bottom or lower primary surface 22 rests on top of an outer surface of the (anterior) vaginal wall 26, as shown in FIGS. 7A and 8.

The curved surface typically curves upward toward the urethra as shown in FIGS. 7A-7C. In other embodiments, the implant 10 can have planar primary surfaces or can include one planar and one curved primary surface. In yet other embodiments, the curvature can be reverse that shown in FIGS. 7A-7C, e.g., the primary surface may project out toward the urethra rather than curve away from the urethra.

The primary surfaces 15 and 22 can be completely smooth to reduce stress on tissue and inhibit complications such as erosion or extrusion. The term “smooth” means that there is a smooth (rather than rough) tactile feel so that its surface finish is non-irritating to adjacent tissue. The primary surfaces 15 and 22 can be continuous solid closed outer surfaces, and, at least for the portions contacting local tissue, may have a constant continuous and uninterrupted line (radius).

In some embodiments, the surface area of the curved middle portion or groove 110 of the first primary surface 15 can be smaller than the surface area of the rear or second primary surface 22. The radius of curvature of surfaces of the outer segments 111 can be greater than that of both 110 and 22 in order to create a disparity between the contact area of the groove (110) and urethra 23, and the contact area of the bottom surface (22) and vaginal wall 26. The two primary surfaces 15, 22 can meet at edges 111e and at outer rounded ends or shoulders 122 (FIG. 3).

In some embodiments, the implant 10 can be a rigid or semi-rigid molded body. The implant 10 can be formed of a biocompatible (inert) material or materials such as a biocompatible polymer(s) or rubber that provides sufficient rigidity to be able to provide the force distribution. In some embodiments, the implant 10 can have a Young's modulus between about 2 and about 10 MPa, substantially corresponding to the range of elasticity between healthy and weakened vaginal wall tissue.

The implant 10 can have a porous body (FIG. 13) or a solid hollow body with a hollow interior 20 (FIG. 11) or combinations of same. The implant 10 can be a monolithic molded body of a biocompatible (non-cytotoxic) material that has a defined three-dimensional shape. In some embodiments, the implant 10 can be a solid, porous and/or hollow monolithic molded body of a plurality of biocompatible materials that define the three-dimensional shape. In some embodiments, the implant 10 comprises polypropylene. In some embodiments, the implant 10 comprises silicone. In some embodiments, the implant 10 is rigid or semi-rigid and comprises a combination of materials and may be formed of a material(s) that is/are radio-opaque or biodegradeable over time, such as, but not limited to, polycaprolactone and poly-L-lactone.

The implant 10 may be coated, impregnated, painted, sprayed, dipped or otherwise formed to include (externally and/or internally) a radio-opaque material, such as barium sulfate, to allow in-vivo imaging. The implant 10 may be coated, impregnated or otherwise formed with a biocompatible (non-cytotoxic) material, such as collagen, to reduce the risk of infection. The implant 10 can incorporate therapeutic agents or drugs that are released to local tissue over time. The term “drug” is used interchangeably with “therapeutic agent” and refers to an agent (e.g., an organic compound, an inorganic compound, a biomolecule, etc.) that has a beneficial effect on a subject/patient, which beneficial effect can be complete or partial. “Biomolecule” as used herein refers to a protein, a polypeptide, a nucleic acid (e.g., a deoxyribonucleic acid and/or a ribonucleic acid), and/or a fragment thereof. Exemplary drugs include, but are not limited to, analgesics such as non-steroidal anti-inflammatory drugs and opioids; antibiotics; anti-scarring agents; steroids; anti-inflammatory agents such as steroids, salicylates, ibuprofen, naproxen, dexamethasone, prednisolone, corticosterone, budesonide, estrogen, sulfasalazine and mesalamine; bisphosphonates; anti-thrombotic agents such as heparin, heparin derivatives, urokinase, and PPack (dextrophenylalanine proline arginine chloromethylketone); antineoplastic/antiproliferative/anti-miotic agents such as paclitaxel, 5-fluorouracil, cisplatin, vinblastine, vincristine, epothilones, endostatin, angiostatin, angiopeptin, and thymidine kinase inhibitors; anesthetic agents such as lidocaine, bupivacaine and ropivacaine; vascular cell growth promoters such as transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); antimicrobial agents such as triclosan, cephalosporins, aminoglycosides and nitrofurantoin; cytotoxic agents, cytostatic agents and cell proliferation affectors; vasodilating agents; antibodies (e.g., monoclonal antibodies and/or polyclonal antibodies); growth factors; cytokines; hormones; vitamins; minerals; or any combination thereof.

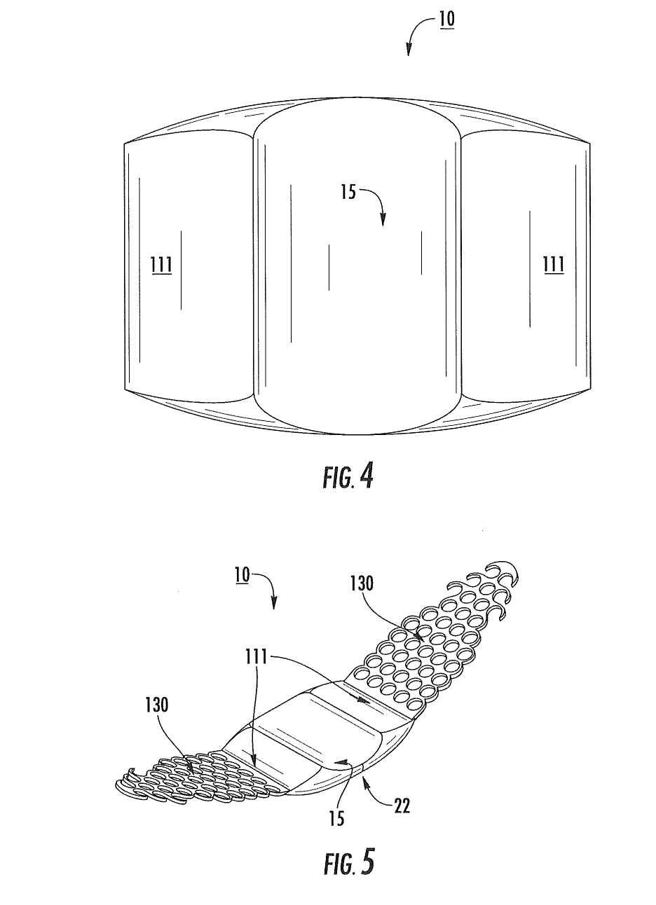

Referring now to FIG. 5, a length or section of macroporous surgical mesh 130 may be optionally attached to both ends 111 of the implant 10. This may encourage tissue integration and improve device fixation over a long period of time. The mesh segments 130 can be configured from any suitable material and may optionally comprise small, interwoven threads of polypropylene. The mesh 130 can be rigid, semi-rigid or flexible. The mesh ends 130 can be tucked into surrounding tissue (rather than locked together). They can be positioned with about a 137 degree angle between them, and can extend towards the obturator membrane.

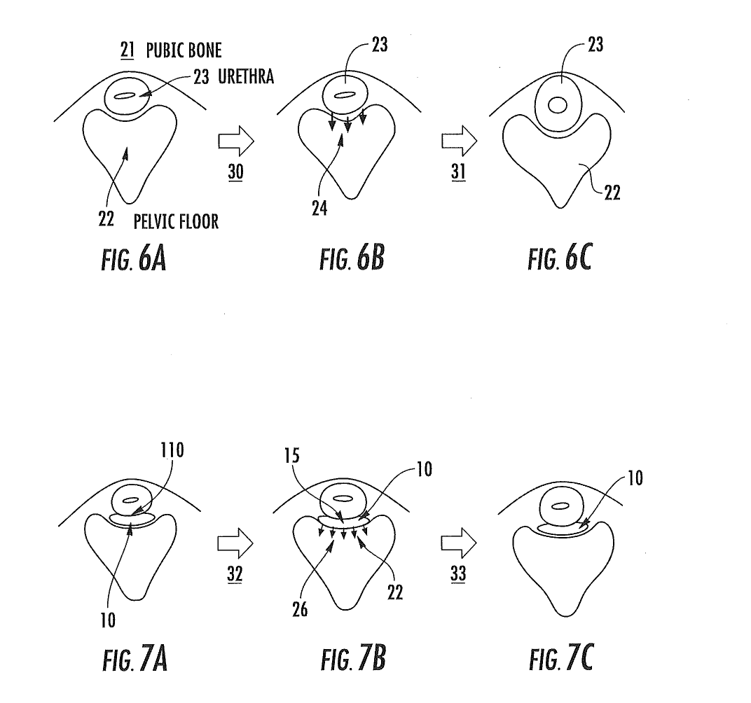

Referring now to FIGS. 6A-6C, a section of the pubourethral anatomy is shown. This anatomy includes a pubic bone 21, urethra 23, and pelvic floor 22 which is comprised of the vaginal wall 26, rectum, and levator ani. FIGS. 6A-6C display the response of a stress incontinent pubourethral system without the invention to a sudden impulse of abdominal pressure 30. Generally stated, the pubic bone 21 is fixed in place, but the increase in intra-urethral force causes the urethra 23 to exert a downward force 24 on the pelvic floor 22. The result 31 can be that the urethra 23 expands and sinks into the pelvic floor 22, thus allowing leakage to occur.

In contrast, as shown in FIGS. 7A-7C, the implant 10, when positioned in the pubourethral anatomy, can inhibit urine leakage. The surface contact area between the urethra 23 and medial portion or groove 110 is smaller than the contact area between the pelvic floor and lower or second primary surface 22. Thus, during an impulse of abdominal pressure 32, the downward force 26 of the urethra is dispersed (distributed) across a greater area and the associated pressure is reduced. The result 33 is that the pelvic floor becomes better able to resist the urethral expansion and inhibit, reduce or prevent leakage. The implant 10 may also optionally be configured to act as a shock absorber to be able to reduce the degree of force transmitted.

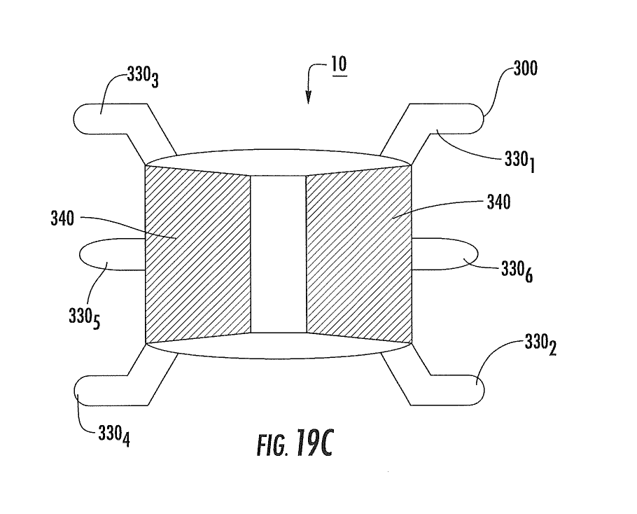

FIG. 8 illustrates the implant 10 in position. The implant 10 can be “free-floating” or self-restraining to be held in position once placed. This is because the holding space that the implant is placed an artificial sub-urethral space due to the surgery (this area is typically filled with fascia and connective tissue). Thus, it is unlikely that the implant 10 will migrate. These two tissue compartments can hold the implant 10 in position without requiring positive fixation. Optionally, sutures or surgical glue may be used to attach the device to the pubourethral fascia to facilitate fixation. Over time, the implant 10 may be encapsulated by fibrous tissue or in-growth which can connect or attach it to the urethra and/or vaginal wall. In some embodiments, the implants 10 can include anti-migration members 300 as will be described further below (see, e.g., FIG. 19A et seq.).

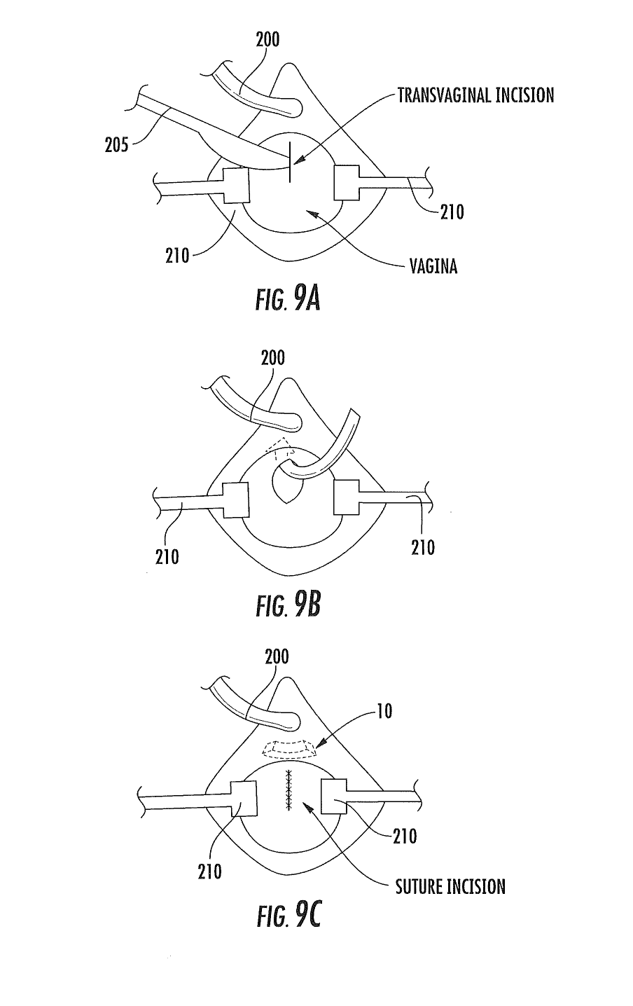

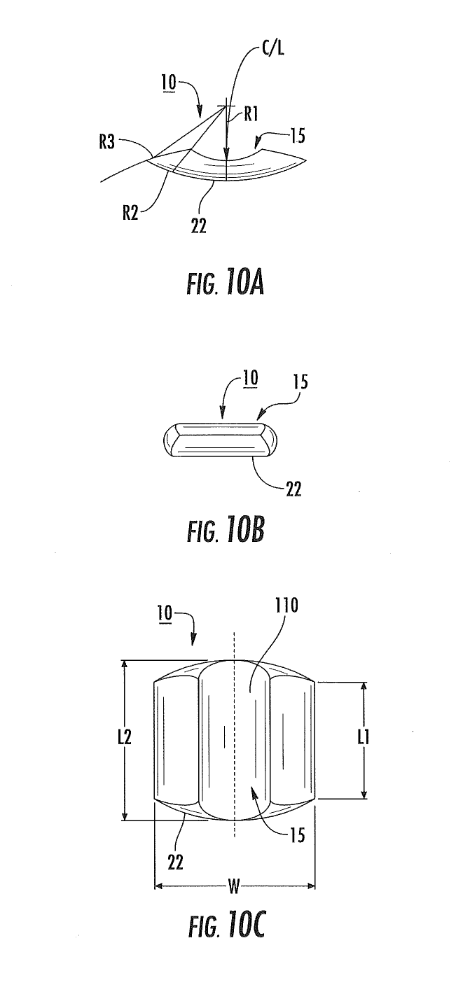

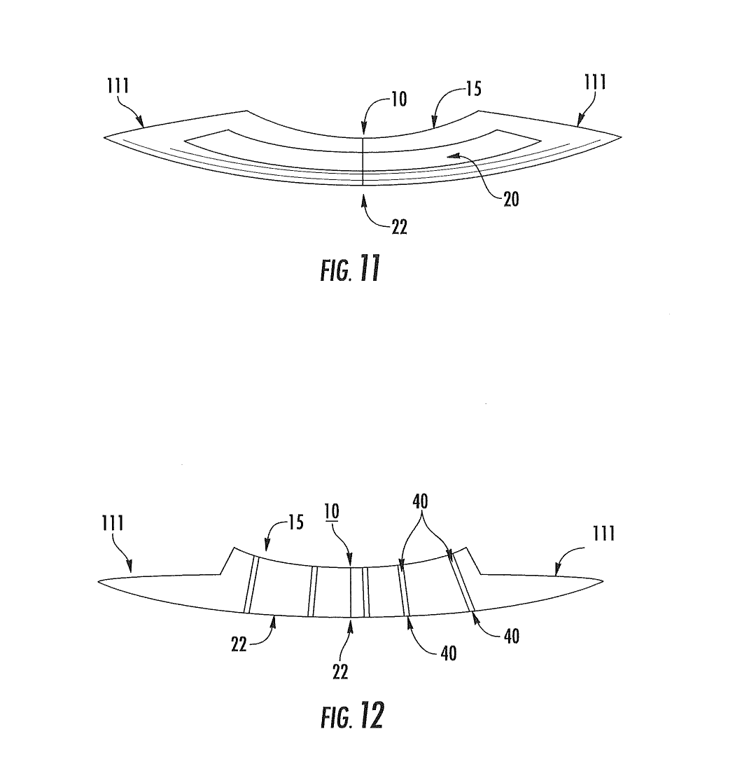

FIGS. 9A-9C illustrate an exemplary sequence of steps for placing the implant 10, typically with a single, transvaginal incision. However, laparoscopic or other surgical pathways can be used. A urethral catheter 200 can be used with clamps 210 to hold the vagina open. The urethra can be palpated with a finger, then the insert can be inserted into the appropriate space between the urethra and vagina wall as shown in FIG. 8. If the implant 10 with mesh ends 130 is placed, then a trochar may be used to place or position the mesh. Ultrasound or another imaging modality may be used to facilitate proper placement or confirm placement of the implant 10.

FIG. 11 illustrates another embodiment of an implant 10. In this embodiment the implant 10 has a hollow interior compartment 20. This compartment 20 may include a different material such as a resilient material or the compartment 20 can include air (non-pressurized). In some embodiments, an internal pressurized bladder may reside in the interior compartment and may comprise saline or other inert fluid. In some embodiments, the implant 10 includes an outer casing that does not require a bladder (it may be sufficiently air or fluid tight) and can have a pressurized compartment of air, saline or other material.

FIG. 12 illustrates that the implant 10 can include a different profile shape on the upper or first primary surface 10.

FIG. 12 also illustrates that the implant 10 can include a plurality of spaced apart apertures 40 (shown as through apertures, but closed ones may also be used). The apertures 40 may allow fluid transfer and/or tissue ingrowth. The apertures 40 can extend in a common direction, top to bottom and/or side to side, and can be parallel or may intersect or be discrete spaced apart channels.

FIG. 13 shows the implant can include pores 40p that may be in a regular or irregular pattern and that may be discrete or interconnected as a matrix to provide channels for fluid flow or exchange through the implant 10. The pores 40p and/or apertures 40a can be sized and configured to allow for white blood cell migration and/or tissue integration. The pores 40p and/or apertures 40 can be configured to provide between about 10-60% porosity, typically between about 10-40% porosity, with aperture and/or pore diameters ranging between about 10 mm to about 300 mm, such as between about 125 mm and 250 mm, including about 125 mm, about 130 mm, about 140 mm, about 150 mm, about 160 mm, about 170 mm, about 180 mm, about 190 mm, about 200 mm, about 210 mm, about 220 mm, about 230 mm, about 240 mm and about 250 mm.

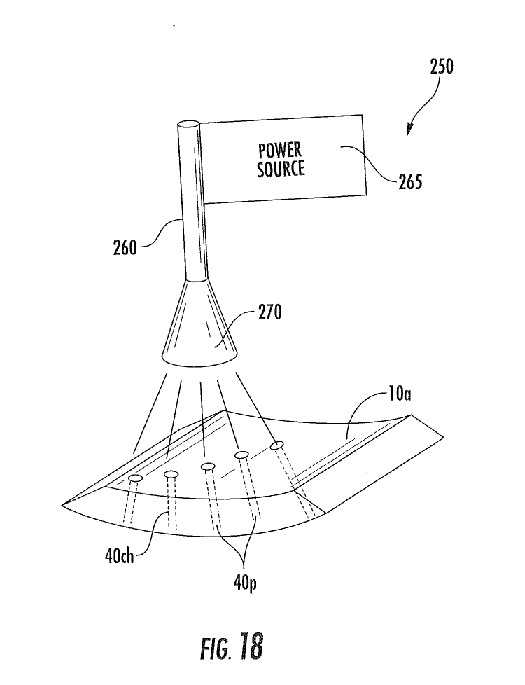

FIGS. 16A and 16B illustrate an exemplary mold 210 that can be used to form the implant 10. The mold 210 includes a mold cavity 210c that has an internal shape that corresponds to the curvature of the implant 10 with defined radii such as discussed with respect to embodiments shown in FIGS. 1, 4, and 10A-10C, for example. The mold 210 can be configured as an injection molding mold 210. The mold 210 can be a single compartment mold or a multiple compartment mold block with discrete mold cavities for fabricating a plurality of discrete implants concurrently. The mold and mold materials may be sterile or aseptic or the mold can be sterilized after it is fabricated.

FIG. 17 illustrates an example of a fabrication system/method 200 that can be used to form a porous implant 10. In this embodiments, a mold material 205 (e.g., silicone) can be mixed with a soluble porogen 208, which is molded together in the mold 210 and integrated into the molded body in a pre-form body 10a. The mold material and porogen can be premixed and introduced into the mold cavity or can be mixed while or after introduced into the mold cavity. The term “porogen” refers to a material that is able to be removed from the primary moldable material after molding in sufficient amounts to create apertures, pores or channels. The pre-form body 10a is then rinsed, sprayed, dipped, chemically reacted or otherwise processed to remove or dissolve away the porogen 208 to thereby form a porous molded body 10b. This can form a sponge-like body with interconnecting pores 40p. The porogen can comprise a salt-leeching material.

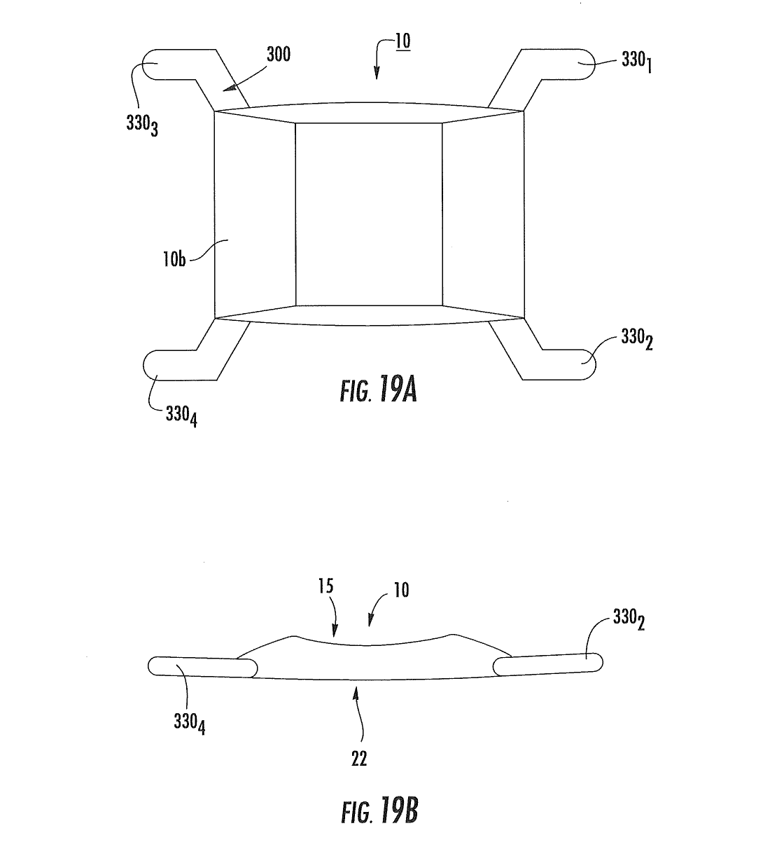

FIG. 18 illustrates a different example of a fabrication system/method 250. Here, the system 250 includes a laser 260 with a laser power source 265 and head 270 that is configured to form pores 40p in a molded body 10a, typically as vertical channels 40ch that extend through the implant 10. The pore channels 40ch can be substantially parallel and may have diameters as described above, e.g., between about typically between about 125 mm and 250 mm.

The methods/systems for forming the porosity described herein are by way of example only and non-limiting to the implants contemplated by the present invention. For example, the mold cavity 210c can include disposable or permanent inserts that extend in the mold to provide the apertures, pores and/or channels 40a, 40p, 40ch (not shown).

FIGS. 19A and 19B illustrate the implant 10 with a plurality of anti-migration members 300 extending outwardly therefrom. In the embodiment shown in FIGS. 19A and 19B, the anti-migration members 300 are arms 330, individually labeled as arms 330₁-330₄ that, at least in position in a body of a patient, can project outward from the implant 10. In some embodiments, the arms 330 can be configured to project outwardly from respective corners as shown in FIGS. 19A and 19B. As shown in FIG. 19B, the arms 330 can reside close to the bottom surface 22 than the top surface 15 (e.g., closer to the anterior wall of the vagina, away from the urethra) in some embodiments. The arms 330 can have a bottom surface that is substantially if not totally co-planar with a portion or all of the primary surface 22 of the implant.

FIG. 19C illustrates that the implant can have one or more additional arms 330₅, 330₆ placed along axially extending sides to project outwardly therefrom, typically in a substantially orthogonal orientation relative to the axial dimension of the implant. FIG. 19C also illustrates that the outer surface can be scored or embossed 340 over a portion or an entire surface thereof, typically over at least a major portion of at least the upper surface, to increase its friction as an anti-migration feature. The scoring or embossment can be in any pattern which may be in a repeating symmetrical pattern or in a non-repeating pattern of any shape, e.g., diamonds, hexagonal and the like. The arms 330 can have a different configuration from that shown. Different arms can have different configurations, shapes or sizes. The side arms 330₅, 330₆ can be smaller or extend outward a lesser distance than one or more of the corner arms 330₁-330₄.

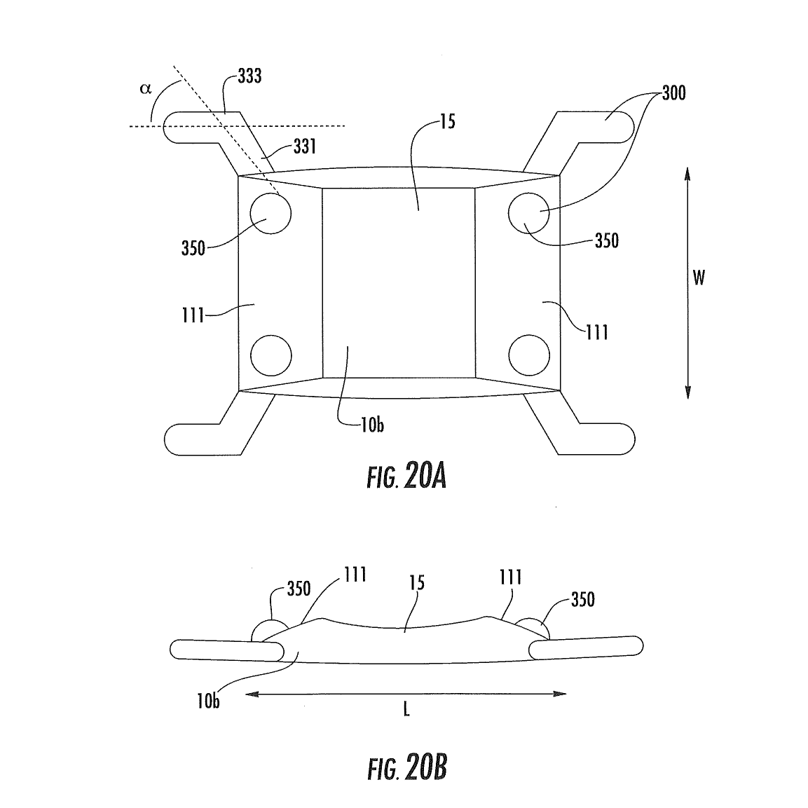

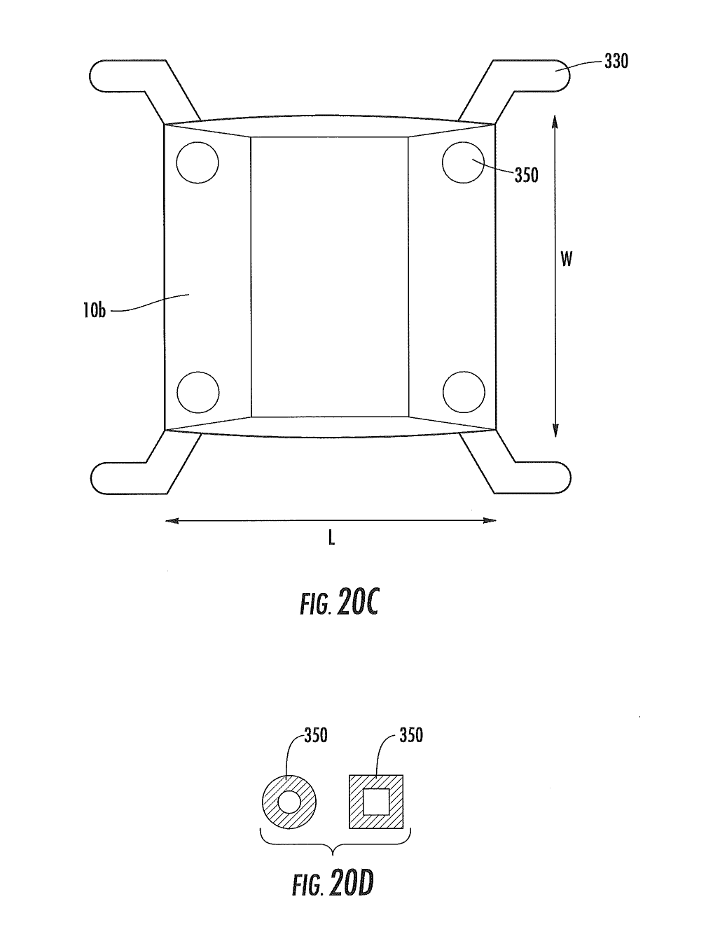

FIGS. 20A and 20B illustrate that the implant can include alternate or additional anti-migration features 300, shown as surface protrusions 350 that can reside on one or more outer surfaces of the implant 10. The surface protrusions 350 can reside proximate each corner of at least a top side of the implant (typically only the top side of the implant on the outer edges thereof). The surface protrusions 350 may be circular as shown or may have other shapes, such as triangular, oval, polygonal, furstoconical, figure 8 shapes and the like, or combinations thereof. The protrusions 350 can be solid and/or have continuous outer surfaces as shown or may be provided as hollow objects and/or shapes with discontinuous surfaces such as a solid perimeter surrounding a recess as shown in FIG. 20D, for example. The perimeter of the protrusion 350 can be continuous or discontinuous. The surface protrusions 350 can be configured to reside on tapered ends 111 as shown in FIGS. 20A, 20B, for example.

As shown in FIG. 20A, for example, in some embodiments, the arms 30 can have a first segment 331 with a length that extends in a first direction then merges into a second free end segment 333. The free end segment 333 can extend at an angle “α” that is between about 15-90 degrees, more typically between about 30-60 degrees, from a centerline of the first segment 331. The free end segment 333 can have a shorter length than the first segment 331. The free end segment 333 can have a longer length than the first segment 331. The free end segment 333 and the first segment 331 can have the same length.

FIG. 20C illustrates that the implant 10 can be configured to have a length L that is equal to or greater than the width W (the width W being in the axial dimension/orientation). The increased length L may inhibit accidental rotation and/or facilitate ease of implantation.

The implant 10 can use any of the different migration features described herein, alone, or any combination.

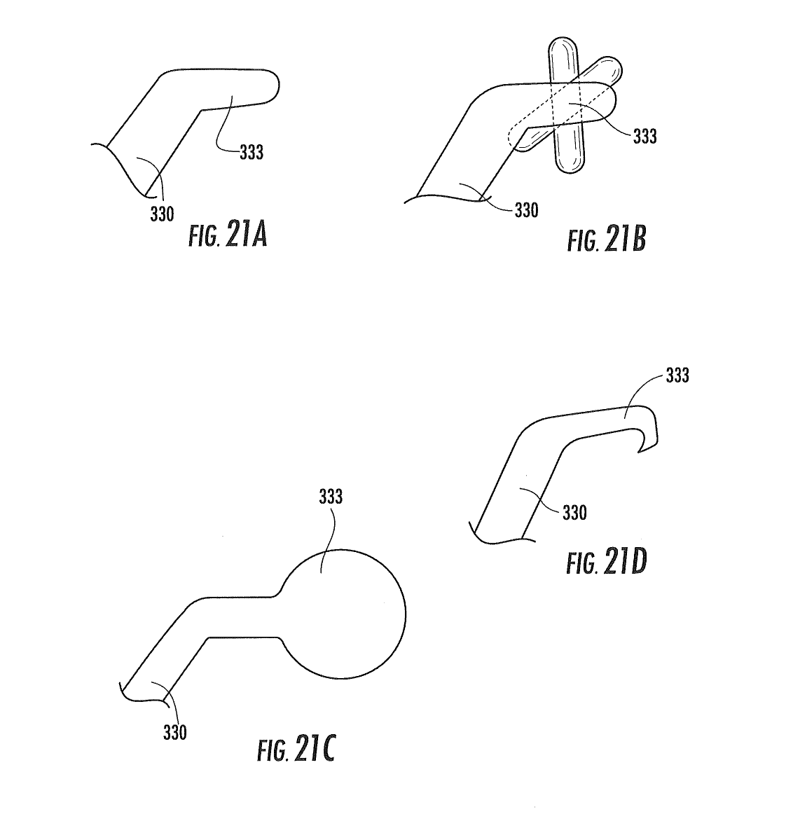

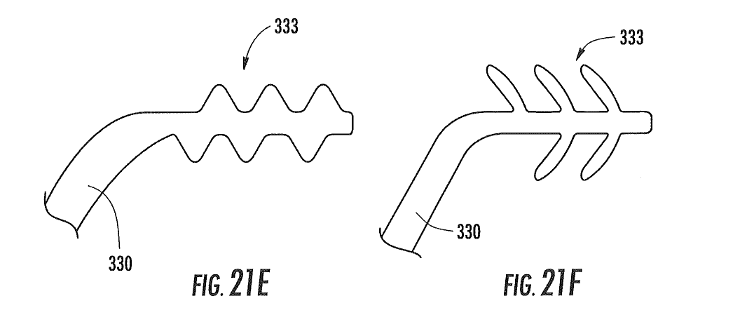

FIGS. 21A-21F illustrate optional configurations for the free end segment 333 of the arms 330. FIG. 21A illustrates a free “straight” end configuration. FIG. 21B illustrates a 3-D cross-shaped configuration with multiple closely spaced ends residing proximate each other. FIG. 21C illustrates a spheroid or bulbous shaped enlarged free end 333. FIG. 21D illustrates a hook configuration. FIG. 21E illustrates the free end 333 can have a spine shape with a series of repeating (e.g., substantially pyramidal) shapes. FIG. 21F illustrates that the free end 333 can have a series of repeating shapes that can be more barb like or have spline segments that angle rearward.

The arms 330 can comprise the same material as the implant body 10b or may be formed of a different material. The arms 330 can be integrally molded to the implant body 10b or be mechanically or chemically (adhesively) attached.

The arms 330 can be semi-rigid arms that have sufficient flexibility to allow for them to bend during implant placement so that the free ends 333 reside closer to the implant body 10b.

The arms 330 can include an elastic material so that the arms 330 automatically deploy into the desired anti-migration configuration upon release from a surgical tool during placement.

The arms 330 can comprise shape memory material. The term “shape memory material” refers to materials that are able to take on a defined pre-formed (expanded) shape after being deformed into a different (collapsed, temporary) shape prior to release from a delivery device. Examples of such materials includes Ni—Ti alloys (such as Nitinol). Other shape memory alloys that may be suitable for medical use include one or more of the following alloys.

| | |
|---|---|
| Titanium-palladium-nickel | Iron-manganese-silicon |
| Nickel-titanium-copper | Nickel-titanium |
| Gold-cadmium | Nickel-iron-zinc-aluminium |
| Iron-zinc-copper-aluminium | Copper-aluminium-iron |
| Titanium-niobium-aluminium | Titanium-niobium |
| Uranium-niobium | Zirconium-copper-zinc |
| Hafnium-titanium-nickel | Nickel-zirconium-titanium |

The term “shape memory materials” also includes shape memory polymers (SMPs) and can include biodegradable SMPs. SMPs are polymeric smart materials that have the ability to return from a deformed state (temporary shape) to their original (permanent) shape induced by an external stimulus, such as temperature change. See, e.g., Lendlein, A., Kelch, S. (2002). “Shape-memory polymers”. Angew. Chem. Int. Ed. 41: 2034-2057; and Lendlein, A., Langer, R. (2002). “Biodegradable, Elastic Shape Memory Polymers for Potential Biomedical Applications”. Science 296 (5573).

Most SMPs can retain two (others can retain three) shapes and the transition between those is induced by the external stimulus, such as temperature. In addition to a temperature-based stimulus, the shape change of SMPs can be triggered by an electric or magnetic field, light exposure (e.g., UV light) or a (gas/liquid) fluid. See, e.g., Mohr, R. et al. (2006), Initiation of shape-memory effect by inductive heating of magnetic nanoparticles in thermoplastic polymers”, Proc. Natl. Acad. Sci. U.S.A. 103 (10): 3540-3545; Lendlein, A. et al. (2005), “Light-induced shape-memory polymers”. Nature 434 (7035): 879-882; and Jinsong Leng, Haibao Lv, Yanju Liu and Shanyi Du. (2008), “Comment on “Water-driven programable polyurethane shape memory polymer: Demonstration and mechanism””. Appl. Phys. Lett. 92: 206105. Lendlein, A., Langer, R. (2002). The contents of the above articles are hereby incorporated by reference as if recited in full herein.

Where the arms 330 include shape memory material, the shape memory material can be encapsulated in an over coat, such as a molded overcoat or may be used alone to form the arms 330. The arms 330 and entire implant 10 are sterilized for medical use.

The surface projections 350 can be integrally molded with the implant body 10b and can comprise the same material as the implant body or a material with a different hardness. If the latter, a two-step molding process may be used to form the projections onto the surface of the implant body. Although not preferred, the surface projections may also be mechanically or chemically (adhesively) attached to the implant body 10b.

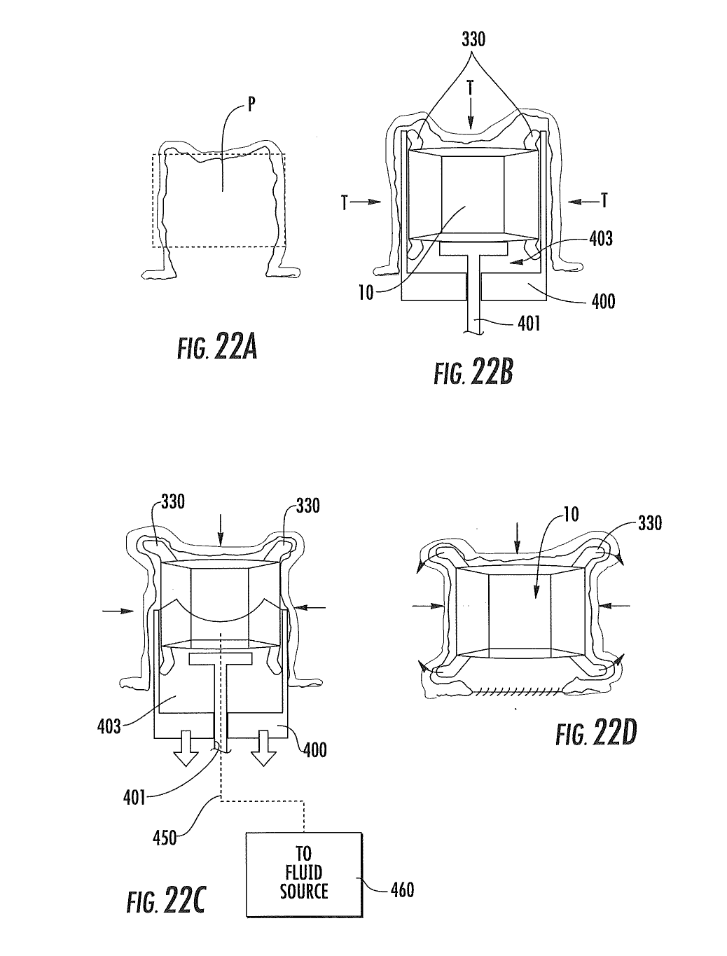

The arms 330 can be configured to reside closer to the body of the implant during placement than in a normal configuration, then resiliently deploy a further distance outward, once in position as shown in FIGS. 22B-22C, for example.

FIGS. 22A-22D show an exemplary sequence of actions that can be used to place the implant 10 using a surgical tool 400. The tool has a cavity 403 that is sized and shaped to hold the implant 10 in a manner that forces the arms 330 closer to the implant body 10b relative to a configuration outside of (post-release or prior to placement in) the tool 400. The tool 400 can include an optional pusher 401 that can be used to force the implant out of the tool cavity. Optionally, however, as shown in FIG. 22D, the tool 400 can include a fluid flow path 450 that introduces pressurized fluid 460 such as fluid comprising saline (with or without a therapeutic agent such as an antibiotic or anti-inflammatory agent) may also or alternatively be used to deploy the implant 10 from the tool cavity 403.

FIG. 22A illustrates that a surgeon can make an initial incision that can create a pocket P between the urethra and anterior vaginal wall. As shown in FIG. 22B, the surgical implantation tool 400 is then inserted into the pocket P in a manner that mechanically extends the pocket while positioning the implant 10 introducing tension T on adjacent tissue above and to the sides of the pocket P (shown by inwardly facing arrows). As shown in FIG. 22C, the tool 400 can be withdrawn while pushing the implant 10 out of the tool cavity 403 and into the pocket P causing the fixation arms 330 to flex outward in response to release from the tool cavity to engage local tissue. FIG. 22D illustrates that all arms 330 are extended to snugly abut and create tension in local tissue (indicated by arrows extending from respective arms 330). The incision can be closed optionally using sutures thereby anchoring the implant 10 in a desired position in the pocket P.

The implants 10 contemplated by embodiments of the invention can have a much smaller area than conventional slings and can optionally be implanted through a single transvaginal incision in a less invasive manner than current incontinence slings. Once fitted under the urethra, the implant 10 will typically not need to be adjusted. There is no tension on the implant itself, so the procedure is much easier to learn and reproduce consistently. Unlike the sling, the implants 10 do not restrict the urethra. The implants 10 can inhibit, reduce or prevent urinary leakage by dispersing or distributing force to inhibit or prevent a sudden expansion associated with stress incontinence. The surfaces of the implant can be smooth and minimize the risk of tissue erosion or organ perforation. Thus, embodiments of the present invention provide urogynecologic implants that reduce, inhibit or prevent stress incontinence by reducing the displacement of the urethra into the pelvic floor during impulses of pressure.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

That which is claimed:

1. A stress incontinence implant, comprising:
a three dimensional rigid or semi-rigid urogynecologic implant body having a defined three dimensional self-supporting shape sized and configured to reside between a urethra and an anterior vaginal wall of a patient, wherein the implant body comprises a plurality of anti-migration members.

2. The implant of claim 1, wherein the anti-migration members include outwardly extending arms.

3. The implant of claim 1, wherein the anti-migration members include surface protrusions.

4. The implant of claim 1, wherein the implant body has at least one embossed primary surface.

5. The implant of claim 1, wherein the anti-migration members include flexible outwardly extending arms that are configured to have sufficient flexibility to flex toward or against the implant body when compressed during placement in the body and sufficient rigidity to translate to a fully extended self-supporting configuration in position in a respective patient.

6. The implant of claim 1, wherein the implant body has four corners, wherein the anti-migration members include at least one flexible outwardly extending arm that resides on each corner, and wherein the arms are configured to have sufficient flexibility to flex toward or against the implant body when compressed during placement in the body and sufficient rigidity to translate to a fully extended self-supporting configuration in position in a respective patient.

7. The implant of claim 5, wherein the arms have a first segment that merges into a second segment that has a free outer end, and wherein the second segment extends at an angle that is between 15-90 degrees with respect to a centerline of the first segment.

8. The implant of claim 1, wherein the implant body comprises four corners, wherein the anti-migration members include surface protrusions at least some of which reside proximate the four corners.

9. The implant of claim 1, wherein the implant body comprises a first primary surface that is adapted to contact an outerwall of the urethra and a second primary surface underlying the first primary surface adapted to contact an anterior vaginal wall, and wherein the anti-migration members comprise arms that reside closer to the second primary surface adjacent the anterior vaginal wall.

10. The implant of claim 1, wherein the implant body and anti-migration members are a molded monolithic body or an integral body with one of the implant body or the anti-migration members having a greater hardness than the other.

11. The implant of claim 1, wherein the implant body has a Young's modulus between about 2 MPa and about 10 MPa

12. The implant of claim 1, wherein the implant body has a width dimension and a length dimension, with the width dimension being about the same or less than the length dimension.

13. The implant of claim 1, wherein the implant body comprises a first primary surface that is adapted to contact an outer wall of the urethra and a second primary surface underlying the first primary surface adapted to contact an anterior vaginal wall, and wherein the first primary surface has medial region that is raised and has an increased thickness relative to outer end portions thereof, and wherein the anti-migration members (i) reside on or extend from the outer end portions thereof or (ii) includes both arms that extend outwardly from the outer end portions and surface protrusions that extend upwardly from the outer end portions.

14. The implant of claim 1, wherein the implant body is sized and configured to surround only about 180 degrees or less of a female urethra and has a maximum thickness that is between about 1 mm to about 5 mm.

15. A surgical tool for placing a urinary stress incontinence implant in a female patient, comprising:
a surgical tool with a cavity; and
a three-dimensional rigid or semi-rigid shaped implant body sized and configured to reside between a urethra and anterior vaginal wall of a female patient, the implant having a plurality of anti-migration arms, wherein the implant body is held in the cavity with the anti-migration aims held proximate the implant body, and wherein the tool is configured to slidably release the implant body from the cavity so that the arms automatically expand outwardly as they exit the tool cavity to engage local tissue.

16. The tool of claim 15, wherein the cavity includes an aperture that slidably receives a pusher, and wherein the pusher is extendable to push the implant body out of the cavity to automatically deploy the arms.

17. A method of fabricating a urinary incontinence implant, comprising:
forming a rigid or semi-rigid three dimensional implant body with anti-migration members, the implant body having sufficient rigidity to define a self-supporting three-dimensional shape with a plurality of attached anti-migration members.

18. The method of claim 17, wherein the forming step is carried out by molding the implant body and the anti-migration members, wherein the anti-migration members include at least one of surface protrusions proximate four corners of a first primary surface or four outwardly extending legs extending off four corners of a second underlying primary surface.

19. A method of implanting a stress urinary incontinence implant, comprising:
providing a surgical tool with a cavity having an open forward end releasably holding an implant body with flexible anti-migration arms extending outwardly from the implant held against sidewalls of the cavity to force the arms toward the implant body; and
releasing the implant body from the cavity of the tool at a pocket location between a urethra and vaginal wall and allowing the arms to return to an outwardly extended configuration to engage local tissue thereat.

20. The method of claim 19, wherein the arms reside on respective corner portions of the implant body, and wherein the implant body can optionally include surface protrusions extending off a first primary surface to face the urethra.
